(19) United States
(12) Patent Application Publication
(10) Pub. No.: US 2022/0047789 A1
(43) Pub. Date: Feb. 17, 2022

(54) ADEQUACY ASSESSMENT METHOD AND SYSTEM

(71) Applicant: Beijing IfmSoft Information Technology Co., Ltd., Beijing (CN)

(72) Inventors: Wei WANG, Beijing (CN); Jia CHEN, Beijing (CN); Yue YANG, Beijing (CN)

(73) Assignee: Beijing IfmSoft Information Technology Co., Ltd., Beijing (CN)

(21) Appl. No.: 17/274,774

(22) PCT Filed: Aug. 28, 2019
(86) PCT No.: PCT/CN2019/102947

§ 371 Date: Mar. 9, 2021

(30) Foreign Application Priority Data
Sep. 10, 2018 (CN) 201811052079.7

(51) Int. Cl. A61M 1/16; G16H 50/30; G16H 10/40; G16H 20/40

(52) U.S. Cl. CPC A61M 1/1619; G16H 50/30; A61M 2202/0498; G16H 20/40; G16H 10/40

(57) ABSTRACT

This application relates to an adequacy assessment method and system. The method includes: acquiring biochemical test data, and extracting the most recent assay data of periodic assay records from the biochemical test data; calculating body fluid volume according to the most recent assay data; calculating a urea clearance index according to the calculated body fluid volume and designated parameters; and outputting the urea clearance index for one adequacy assessment by display.

### CROSS-REFERENCE TO RELATED APPLICATIONS

This application is a National Stage of International Application No. PCT/CN2019/102947, filed on Aug. 28, 2019, which claims priority to Chinese Patent Application No. 201811052079.7, filed on Sep. 10, 2018, both of which are hereby incorporated by reference in their entireties.

### TECHNICAL FIELD

This application relates to the field of medical data, and in particular to an adequacy assessment method and system.

### BACKGROUND

Renal disease is a major chronic disease that seriously endangers human health and is usually treated by renal replacement therapy (RRT). RRT is a therapy where water and solutes are continuously and slowly removed from blood usually through cardiopulmonary bypass (CPB) and hemodialysis (HD), which serves a kidney function to minimize the impact of changes in the concentration of solutes and volume in blood on the body. Adequacy assessment of dialysis in RRT is an important method to ensure the quality of treatment. In an RRT course for patients with renal diseases such as uremia, dialysis adequacy must be ensured to improve the life quality of maintenance hemodialysis (MHD) patients. Dialysis adequacy is an important factor affecting the prognosis of end-stage renal disease patients. Data from large international, multi-center HD studies shows that adequate HD treatment for end-stage renal disease patients can reduce the morbidity and mortality of complications. Adequacy assessment of HD is an important method to improve dialysis and ensure the quality of dialysis.

At present, an actual method for adequacy assessment of HD in China is an assessment method where a blood sample is usually collected to detect a urea clearance index, but assessment is generally made once every 1 month to 3 months clinically, resulting in a lack of timeliness, if real-time assessment is required, multiple blood samples need to be repeatedly collected for biochemical tests. Considering that the frequent testing increases burdens on both medical insurance and patients and frequent blood drawing is also inappropriate for anemia patients, it is impossible to monitor dialysis adequacy or adjust a treatment regimen in real time during a dialysis process.

In the prior art, a Kt/V parameter after dialysis is predicted through an algorithm model with measured relevant parameters of used dialysate, which is then compared with a Kt/V parameter after actual dialysis. However, an algorithm provided in the prior art is based on the measurement data of the used dialysate and lacks the direct blood data of a patient. Therefore, a predicted Kt/V parameter cannot reflect the actual conditions of the patient. There is large error between an actual Kt/V index after treatment and a predicted target Kt/V index, and an expected dialysis treatment effect cannot be accurately achieved. In order to make an actual measured Kt/V value and a set target Kt/V value tend to be consistent, it is necessary to compare the actual measured Kt/V index with the target Kt/V index and continuously make trial-and-error adjustments based on experience with reference to previous blood flow and dialysate flow values during a subsequent dialysis process. It often needs to be repeated many times, requires complicated steps, leads to inaccurate results, and cannot adapt to continuous changes of the patient's body. The measurement of dialysate used by the patient also requires special sensor devices that are expensive. If a dialyzer is not equipped with the sensor devices, the measurement of dialysate cannot be achieved.

An existing patent discloses a device that can predict a target Kt/V value through the actual physiological parameters of a patient and can also automatically adjust blood flow and dialysate flow through a controller, so that an actual measured Kt/V value can quickly and accurately be consistent with the target Kt/V value, thus achieving expected dialysis treatment effect. However, it is found from the clinical practice that although the above-mentioned device can automatically adjust blood flow and dialysate flow through a controller, it is complicated to implement and has high requirements on devices and procedures. Various sophisticated and complex controllers and the like are required, which results in a high cost and difficult promotion and application in a short time. Moreover, an implementation process involves quite a lot of automatic control devices, so it is difficult to achieve flexible intervention clinically and clinical operations are inconvenient.

### SUMMARY

In view of the above analysis, this application is intended to provide an adequacy assessment method and system, which solves the problems in the prior art that the dialysis adequacy cannot be monitored in real time through repeated blood sampling during a dialysis process, a predicted urea clearance index cannot, reflect the actual conditions of a patient, expected dialysis treatment effect cannot be accurately achieved, and devices currently used for dialysis monitoring are complicated and costly.

The objective of the present application is achieved by the following technical solutions.

In one aspect, an embodiment of the present application provides an adequacy assessment method including: acquiring biochemical test data, and extracting the most recent assay data of periodic assay records from the biochemical test data calculating body fluid volume according to the most recent assay data; calculating a urea clearance index according to the calculated body fluid volume and designated parameters; and outputting the urea clearance index for one adequacy assessment by display.

Based on an embodiment of the above method, an adequacy assessment result for each dialysis and corresponding diagnosis and treatment data are stored in a database.

Optionally, the acquiring biochemical test data may include: inputting biochemical test data through an input interface or automatically acquiring biochemical test data from a medical system of biochemical test data.

Optionally, the calculating body fluid volume according to the most recent assay data may include: according to periodic assay data and dialysis data on the day of the assay, acquiring blood urea concentration, dialysis time, removal volume of water, blood flow, and urea clearance rate at the beginning and end of dialysis; setting an assumed value for initial body fluid volume, and after the dialysis starts, calculating urea concentration per unit time until the dialysis ends to obtain urea concentration at the body fluid volume; and continuously adjusting the assumed value of body fluid volume, so that the urea concentration calculated is consistent with urea concentration tested at the end of the dialysis to obtain actual body fluid volume.

Optionally, the calculating the urea clearance index according to the body fluid volume calculated and designated parameters may include: according to known actual body fluid volume at the beginning of dialysis, obtaining water volume distributed in visceral organs with high and low blood flow, and according to the removal volume of water per unit time, deriving water volume in each visceral organ after each unit time; and obtaining urea concentration in arterial blood at the beginning of dialysis through actual assay, assuming that urea concentration in various visceral organs and arterial blood at the beginning of dialysis are consistent, and continuously and sequentially calculating urea concentration in various visceral organs and blood after each unit lime according to the following formula until urea concentration in visceral organs with high and low blood flow and arterial blood at tire end of dialysis are calculated:

$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )} ;$$

$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )} ;$$

$${C}_{A} ( t + 1 ) = \frac{{Q}_{A} \times {C}_{A} ( t ) - K \times {C}_{A} ( t )}{{Q}_{A}} ;$$

where C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} On is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

Optionally, the urea clearance index may be calculated based on a ratio of urea concentration in arterial blood at the end and beginning of the dialysis.

Beneficial effects of the technical solution: the present application discloses an adequacy assessment method, including: acquiring biochemical test data, and extracting the most recent assay data of periodic assay records from the biochemical test data; calculating body fluid volume according to the most recent assay data; calculating a urea clearance index according to the calculated body fluid volume and designated parameters; and outputting the urea clearance index for one adequacy assessment by display. Compared with the prior art, the method of the present application needs not to collect blood from a patient at multiple times during a dialysis treatment process, realizes real-time monitoring of dialysis adequacy only through an algorithm software tool, can accurately achieve expected dialysis treatment effect and also solves the problem that existing dialysis devices are complex and costly. This application acquires biochemical test data of a patient before dialysis at one time to realize automatic assessment of each dialysis, which does not rely on multiple biochemical tests of blood sampling before and after HD and does not increase the blood loss burden of a patient due to repeated blood sampling. The dialysis adequacy can be monitored in real time and a treatment regimen can be adjusted during a dialysis process.

In another aspect, an embodiment of tire present application provides an adequacy assessment system including: an input device, a processor for calculating and an output device. The input device is configured to acquire biochemical test data and extract the most recent assay data of periodic assay records from the biochemical test data; the processor for calculating is configured to calculate body fluid volume according to the most recent assay data and thus calculate a urea clearance index according to the calculated body fluid volume and designated parameters; and the output device is configured to output the urea clearance index for one adequacy assessment by display.

Based on another embodiment of the above system, the system may further include a storage configured to store adequacy assessment result for each dialysis and corresponding diagnosis and treatment data.

Optionally, the input device may be specifically configured to input biochemical test data through an input interface or automatically acquire biochemical test data from a medical system of biochemical test data.

Optionally, the processor for calculating may be specifically configured to calculate body fluid volume according to the most recent assay data; calculate urea concentration at the end of dialysis according to the calculated body fluid volume, dialysis time, removal volume of water, blood flow, and urea clearance rate of a dialyzer; and calculate the urea clearance index based on a ratio of urea concentration in arterial blood at the end and beginning of dialysis.

Other features and advantages of the present application will be described in the following specification, and some of these will become apparent from the specification or be understood by implementing the present application. The objectives and other advantages of the present application can be implemented or obtained by structures specifically indicated in the written specification, claims, and accompanying drawings.

### BRIEF DESCRIPTION OF DRAWINGS

The accompanying drawings are provided merely for illustrating specific embodiments and are not considered as limiting the present application. Throughout the accompanying drawings, the same reference numerals represent the same components.

FIG. 1 is a flow chart of an adequacy assessment method provided according to an embodiment of the present application;

FIG. 2 is an algorithm flow chart of the adequacy assessment method provided according to an embodiment of the present application;

FIG. 3 is a schematic structural diagram of an adequacy assessment system provided according to an embodiment of the present application;

FIG. 4 is a schematic diagram of an output interface of the dialysis adequacy assessment system provided according to an embodiment of the present application; and

FIG. 5 is a calculation flow chart of an algorithm model provided according to an embodiment of the present application.

### DESCRIPTION OF EMBODIMENTS

Preferred embodiments of the present application will be described in detail below with reference to the accompanying drawings. The accompanying drawings constitute a part of the present application, and are used together with the embodiments of the present application for explaining principles of the present application rather than for limiting a scope of the present application.

At present, an actual method for adequacy assessment of HD in China is an assessment method where blood samples are usually collected to detect a urea clearance index, thus there are the following defects; biochemical tests by blood samples collected multiple times, and burdens on both medical insurance and patients increased due to the frequent testing. Moreover, a Kt/V parameter after dialysis is predicted through an algorithm model with measured relevant parameters of used dialysate and then compared with a Kt/V parameter after actual dialysis. There are the following problems: large error exists between an actual Kt/V index after treatment and a predicted target Kt/V index, expected dialysis treatment effect cannot be accurately achieved, and dialysis devices are expensive. The method and system of the present application are provided to solve deficiencies and problems in current dialysis adequacy assessment. The application acquires biochemical test data of the patient before dialysis at one time to realize automatic assessment of each dialysis, which does not rely on multiple biochemical tests of blood sampling before and after HD and does not increase the blood loss burden of the patient due to repeated blood sampling. The dialysis adequacy can be monitored in real time and a treatment regimen can be adjusted during a dialysis process, without relying on a dialyzer and peripheral accessory devices. The application is suitable for alt kinds of dialyzers to assess dialysis regimens and treatment effects on patients.

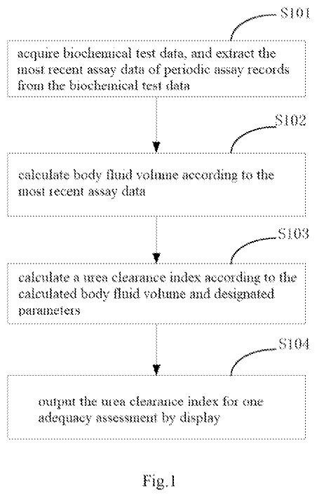

According to a specific embodiment of the present application, an adequacy assessment method is disclosed, as shown in FIG. 1, including the following steps:

S101: acquiring biochemical test data, and extracting the most recent assay data of periodic assay records from tire biochemical test data;

S102: calculating body fluid volume according to the most recent assay data;

S103; calculating a urea clearance index according to the calculated body fluid volume and designated parameters; and

S104: outputting the urea clearance index for one adequacy assessment by display.

As shown in FIG. 1, the adequacy assessment method according to an embodiment of the present: application includes; acquiring biochemical test data, and extracting the most recent assay data of periodic assay records from the biochemical test data; calculating body fluid volume according to the most recent assay data; calculating a urea clearance index according to the calculated body fluid volume and designated parameters; and outputting the urea clearance index for one adequacy assessment by display. Compared with the prior art, the present application solves the problems that repeated blood sampling and frequent assays in a current dialysis treatment process increases the physical and economic burden of a patient, and also avoids the problems that an existing dialysis algorithm model cannot accurately predict the dialysis adequacy in real, time and related devices used are complicated and expensive.

In a specific embodiment of the present application, an adequacy assessment result for each dialysis and corresponding diagnosis and treatment data are stored in a database. That is, a database of a medical system stores large amounts of historical diagnosis and treatment data of patients, provides algorithm software with experience historical data and model algorithms for assessment and calculation, and stores an adequacy assessment result for each dialysis of patients. That is, dialysis treatment and periodic biochemical assay data of long-term HD patients are acquired and analyzed to provide doctors with assessment methods and algorithms for required key assessment indexes, thus assisting doctors in realizing the correct treatment and the optimal prognostic assessment for patients.

It should be noted that, if a patient is subjected to dialysis for the first time, large amounts of diagnosis and treatment data of the patient stored in the database, including; name, gender, age, body height, dialysis history, body weight, dialysis time, biochemical, test data, dialysis information data, and the tike, need to be used to screen out a first-dialysis regimen suitable for the patient through comparison and determination as a reference for medical staff, such as selection of dialyzers for different urea clearance rates and determination of a total removal volume of water during dialysis.

In a specific embodiment of the present application, the acquiring biochemical test data may include: inputting biochemical test data through an input interface or automatically acquiring biochemical test data from a medical system of biochemical test data. In other words, the acquiring biochemical test data can refer to inputting clinical data of patients by medical staff or automatically acquiring clinical data of patients by a computer.

It should be noted that the biochemical test data values acquired manually or automatically may include: test date, urea nitrogen before dialysis, urea nitrogen after dialysis, creatinine before dialysis, creatinine after dialysis, sodium before dialysis, sodium after dialysis, phosphorus before dialysis, phosphorus after dialysis, potassium before dialysis, potassium after dialysis, and the like; and the dialysis parameter information data acquired manually or automatically may include: dialysis date, dialysis duration, blood flow rate, dialysate flow rate, body weight after the last dialysis, body weight before dialysis, body weight after dialysis, urea clearance rate of a dialyzer, and foe like.

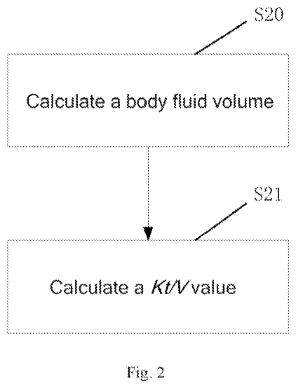

FIG. 2 is an algorithm flow chart of the adequacy assessment method according to an embodiment of the present application.

At present, the second-generation formula proposed by Daugirdas in 1993 is most widely used clinically to calculate a single-pool Kt/V (spKt/V) based on a single-pool urea kinetic model. A natural logarithm formula of Kt/V:Kt/V=−Ln(R−0.008×t)+(4−3.5×R)×UF/W, where Ln is the natural logarithm; R is (BUN after dialysis)/(BUN before dialysis); t is the time of one dialysis, expressed, in hour; UF is an ultrafiltration volume, expressed in Liter; W is a body weight of a patient after dialysis, expressed in kilogram; and BUN is urea nitrogen content, namely, urea concentration.

The present application provides a novel improved algorithm based on the single-pool urea kinetic model, called Schuneditz's local urea kinetic model, which avoids the need to obtain BUN urea concentration by blood drawing and assay during each, dialysis to calculate Kt/V. The urea clearance index, namely, Kt/V, is a ratio of urea volume removed by a dialyzer to total urea volume in the body, where K represents urea clearance rate (L/h) of a dialyzer, which is recorded in a product specification of the dialyzer; t is a single-dialysis time (h); and Vis volume of distribution of urea in the body (L), namely, total water volume in the human body, which is calculated from body weight, body height, and body surface area (BSA). The Kt product reflects a urea clearance amount by single dialysis, and Kt/V reflects a ratio of the urea clearance amount by single dialysis to the total urea amount in toe patient's body.

Schuneditz's local urea kinetic model is based on the following observations: there are visceral organs with high water content and low blood volume (visceral organs with low blood flow: muscle, bone, skin, fat tissue, etc.) and visceral organs with low water content and high blood volume (visceral organs with high blood flow; heart, brain, digestive organ, lung, etc.) in the human body. The water content in the human body is referred to as body fluid volume.

With the Schuneditz's local urea kinetic model, a formula for calculating urea concentration per unit interval can be obtained through urea concentration in visceral organs with low and high blood flow and arterial blood at any time t, where the unit interval can be set to 1 min according to actual clinical application requirements.

In the Schuneditz's local urea kinetic model, blood volume flowing out from the heart is represented by Q_{A}, and blood volume flowing back to the heart is represented by Q_{F}. A difference between the blood volume flowing out from the heart and the blood volume flowing back to the heart is blood volume flowing to various visceral organs. Blood flow in visceral organs with high blood flow is represented by Q_{H}, and blood flow in visceral organs with low blood flow is represented by Q_{L}. During the whole process of dialysis, in the Schuneditz's local urea kinetic model, V_{H} represents water content in visceral organs with high blood flow, and V_{L} represents water content in visceral organs with low blood flow. V_{H}(t) represents water content in visceral organs with high blood flow at time t. V_{H}(t+1) represents water content in visceral organs with high blood flow at time t+1. V_{L}(t) represents water content in visceral organs with low blood flow at time t. V_{L}(t+1) represents water content in visceral organs with low blood flow at time t+1. Moreover, removal volume of water per unit time is represented by F, which is calculated based on a total removal volume of water by one dialysis, and thus water content in each visceral organ at time t+1 can be obtained from water content in each visceral organ at time t.

A change of a urea content in a visceral organ with high blood flow at any time point in dialysis is equal to a difference between urea amount per unit time in blood flowing into the visceral organ with high blood flow and urea amount per unit time in blood flowing out from the visceral organ. Urea concentration in arterial blood flowing into visceral organs with high blood flow is represented by GA(t), urea concentration in visceral organs with high blood flow is represented by C_{H}(t), and the high blood flow is represented by Q_{H}. In this model, it is assumed that blood flow per unit time flowing into visceral organs with high blood flow is constant, and urea concentration in blood flowing out from visceral organs with high, blood flow is consistent with urea concentration in visceral organs with high blood flow. The following formula can be obtained through the model described above:

$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$

Similarly, if urea concentration in visceral organs with low blood flow is represented by C_{L}(t), and the low blood flow is represented by Q_{L}, the following formula can be obtained:

$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$

Urea amount in visceral organs with high blood flow can be obtained by multiplying urea concentration in visceral organs with high blood flow with water volume in visceral organs with high blood flow. Urea amount in visceral organs with low blood flow can be obtained by multiplying urea concentration in visceral organs with low blood flow with water volume in visceral organs with low blood flow. A change of the total urea amount in visceral organs with high and low blood flow over time t is equal to urea amount removed by nitration of a dialyzer; and a change of the urea concentration in arterial blood flowing into visceral organs with high or low blood flow over time t is also equal to urea amount removed by filtration of the dialyzer, that is, urea change in visceral organs with high and low blood flow, urea change in arterial blood, and urea change caused by filtration of the dialyzer are the same. If urea clearance rate of the dialyzer is represented by K and blood flow is represented by Q_{A}, the following formula is obtained:

$${C}_{A} ( t + 1 ) = \frac{{Q}_{A} \times {C}_{A} ( t ) - K \times {C}_{A} ( t )}{{Q}_{A}}$$

In a specific embodiment of the present application, the calculating body fluid volume according to the most recent assay data may include: according to periodic assay data and dialysis data on the day of the assay, acquiring blood urea concentration, dialysis in time, removal volume of water, blood flow, and urea clearance rate at the beginning and end of dialysis; setting an assumed value for initial body fluid volume, and after the dialysis starts, calculating urea concentration per unit time until the dialysis ends to obtain urea concentration at the body fluid volume; and continuously adjusting the assumed value of body fluid volume, so that calculated urea concentration is consistent with urea concentration tested at the end of the dialysis to obtain actual body fluid volume.

In another specific embodiment of the present application, the urea clearance index calculated according to the calculated body fluid volume and designated parameters may include: according to known actual body fluid volume at the beginning of dialysis, obtaining water volume distributed in visceral organs with high and low blood flow, and according to removal volume of water per unit time, deriving water volume in each visceral organ after each unit time; and obtaining urea concentration in arterial blood at the beginning of dialysis through actual test, assuming that urea concentration in various visceral organs and arterial blood, at the beginning of dialysis are consistent, and continuously and sequentially calculating urea concentration in various visceral organs and blood after each, unit time according to the following formula until urea concentration in visceral organs with high and low blood flow and arterial blood at the end of dialysis are obtained:

$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$

$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$

$${C}_{A} ( t + 1 ) = \frac{{Q}_{A} \times {C}_{A} ( t ) - K \times {C}_{A} ( t )}{{Q}_{A}}$$

where, C_{H}(t) is urea concentration in visceral organs with high blood flow at time t, C_{L}(t) is urea concentration in visceral organs with Sow blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, Q_{L}(t) is water volume in visceral organs with low blood flow at time t, and K is urea clearance rate of a dialyzer.

It should be noted that, as shown in the algorithm flow chart in FIG. 2, the algorithm of the present application is mainly completed in two steps: calculating body fluid volume of a patient by using periodic assay data and using the body fluid volume to calculate a Kt/V value on any day under various dialysis conditions.

S20, body fluid volume is calculated: according to periodic biochemical assay data and dialysis data of a patient on the day of the assay, actual values of all parameters including blood urea concentration, dialysis time, removal volume of water, blood flow, urea clearance rate, and the tike except body fluid volume at the beginning and end of dialysis can be acquired; an assumed value is set for initial body fluid volume, and after the dialysis starts, urea concentration per 1 min is calculated according to a formula until the dialysis ends to obtain calculated urea concentration at the body fluid volume; and the assumed value of body fluid volume is continuously adjusted to obtain body fluid volume value making calculated urea concentration consistent with tested urea concentration. The input parameters and output results of body fluid volume calculation are shown in Table 1 below:

| | |
|---|---|
| Patient name | — |
| Dialysis time (I ) | Input |
| Blood flow (mL/min) | Input |
| Dialysate flow (mL/min) | Input |
| Total removal volume of water (L) | Input |
| Body weight atler dialysis (kg) | Input |
| Urea clearance rate (mL/min) | Input |
| BUN before dialysis (mg/dL) | Input |
| BUN after dialysis (mg/dL) | Input |
| Body fluid volume (mL) | Output |
| Body weight after dialysis when the body fluid | Output |
| volume is determined (kg) | |

S21, A Kt/V is calculated: body fluid volume is calculated using the most recent periodic assay data; urea concentration at the beginning of dialysis is assumed to be 1 mg/mL, and urea concentration at the end of dialysis is calculated based on dialysis time, removal volume of water, blood flow, and a urea clearance coefficient of a dialyzer; a ratio of urea concentration before and after dialysis is obtained; and finally, the ratio of urea concentration before and after dialysis is substituted into the Daugirdas formula to obtain a Kt/V value. The input parameters and output results of Kt/V value calculation are shown in Table 2 below:

| | |
|---|---|
| Patient name | — |
| Dialysis time (h) | Input |
| Dialysate flow (mL/min) | Input |
| Blood flow (mL/min) | input |
| Body fluid volume (mL) | Input |
| Body weight after dialysis when the body | Input |
| fluid volume is determined (kg) | |
| Body weight before dialysis (kg) | Input |
| Body weight gain (kg) | Input |
| Urea clearance rate (mL/min) | Input |
| Kt/V | Output |

Specific steps of the above algorithm process are as follows;

S201: water volume distributed in visceral organs with high and low blood flow are calculated according to the following formulas:

V_{H}(t)=0.2×V_{T}(t)

V_{L}(t)=0.8×V_{T}(t)

where, V_{T}(t) is body fluid volume at time t, V_{H}(t) is water content in visceral organs with high blood flow at time t, and V_{L}(t) is water content in visceral organs with low blood flow at time t.

S202: water content in each visceral organ at time t+1 is derived from water content in each visceral organ at time t according to the following formulas:

V_{H}(t+1)=V_{H}(t)−0.2×F

V_{L}(t+1)=V_{L}(t)−0.8×F

where, F=V_{T}(t+1)−V_{T}(t) represents removal volume of water per unit time from time t to time t+1.

S203, urea concentration in arterial blood at the beginning of dialysis is obtained through actual test, it is assumed that urea concentration in various visceral organs and arterial blood at the beginning of dialysis are consistent, and urea concentration in various visceral organs and blood after each unit time are continuously and sequentially calculated according to the following formula until area concentration in visceral organs with high and low blood flow and arterial blood at the end of dialysis are obtained:

$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$

$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$

$${C}_{A} ( t + 1 ) = \frac{{Q}_{A} \times {C}_{A} ( t ) - K \times {C}_{A} ( t )}{{Q}_{A}}$$

where, C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood, flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

In a specific embodiment of the present application, a urea clearance index is calculated based on a ratio of urea concentration in arterial blood at tire end and beginning of the dialysis. That is, the ratio of urea concentration in arterial blood at the end and beginning of the dialysis is substituted into the following urea clearance index calculation formula:

Kt/V=−Ln(R−0.008×t)+(4−3.5×R)×UF/W

where, Ln is the natural logarithm: R is (BUN after dialysis)/(BUN before dialysis); t is the time of one dialysis, expressed in hour; UF is ultrafiltration volume, expressed in Liter; W is body weight of a patient after dialysis, expressed in kilogram; and BUN is urea nitrogen content, namely, urea concentration.

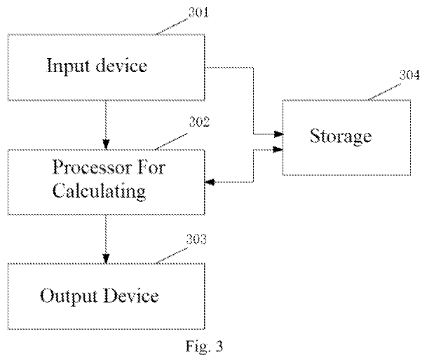

An embodiment of the present application provides an adequacy assessment system, which belongs to the same technical concept as the above adequacy assessment method. FIG. 3 is a schematic structural diagram of an adequacy assessment system according to an embodiment of the present application.

As shown in FIG. 3, the adequacy assessment system of the embodiment of the present application includes: an input device 301, a processor for calculating 302, and an output device 303. The input device 301 is configured to acquire biochemical test data and extract the most recent assay data of periodic assay records from the biochemical test data; the processor for calculating 302 is configured to calculate body fluid volume according to the most recent assay data and thus calculate a urea clearance index according to the calculated body fluid volume and designated parameters; and the output device 303 is configured to output the urea clearance index for one adequacy assessment by display. The system uses algorithm software to acquire biochemical test data of a patient before dialysis at one time to realize automatic assessment of each dialysis, which does not rely on multiple biochemical tests of blood sampling before and after HD and does not increase the blood loss burden of a patient due to repeated blood sampling. The dialysis adequacy can be monitored in real time and a treatment regimen can be adjusted during a dialysis process, without relying on a dialyzer and peripheral accessory devices. The system is suitable for all kinds of dialyzers to assess dialysis regimens and treatment effects on patients.

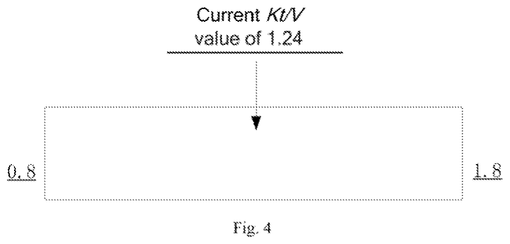

It should be noted that the input device 301 is configured for medical staff to input or a computer to automatically acquire clinical data of patients. The processor for calculating 302, namely, an algorithm model module, is configured to process received clinical data of a patient. Dialysis adequacy assessment result of a patient is obtained through the calculation of an adequacy assessment algorithm model. A strip chart illustrating the dialysis adequacy of a patient is output through the output device 303, namely, the schematic diagram of an output interface of the dialysis adequacy assessment system shown in FIG. 4. In the figure, the dialysis adequacy status showing in actual dialysis result of a patient is visually illustrated. In the dialysis adequacy assessment system of the embodiment of the present application, index values output after the clinical assessment of the obtained biochemical test data by the mathematical model algorithm can be seen in FIG. 4.

In a specific embodiment of the present application, as shown in FIG. 3, the system also includes a storage 304 configured to store adequacy assessment result for each dialysis and corresponding diagnosis and treatment data. The storage 304 is configured to store large amounts of historical diagnosis and treatment data of patients, provide the processor for calculating 302 with experience historical data and model algorithms for an algorithm model module to assess and calculate, and store adequacy assessment result for each dialysis of patients. That is, dialysis treatment and periodic biochemical assay data of long-term HD patients are acquired and analyzed to provide doctors with assessment methods and algorithms for required key assessment indexes, thus assisting doctors in realizing the correct treatment and the optimal prognostic assessment for patients.

In actual clinical applications, if a patient is subjected to dialysis for the first time, an algorithm model module uses large amounts of diagnosis and treatment data of the patient in the storage (including: name, gender, age, body height, dialysis history, body weight, dialysis time, biochemical test data, dialysis information data, and the like) to screen out a first-dialysis regimen suitable for the patient through comparison and determination as a reference for medical staff.

In a specific embodiment of the present application, the input device is specifically configured to input biochemical test data through an input interface or automatically acquire biochemical test data from a medical system of biochemical test data. More specifically, the input device is configured to acquire biochemical test data values (including: test date, urea nitrogen before dialysis, urea nitrogen after dialysis, creatinine before dialysis, creatinine after dialysis, sodium before dialysis, sodium after dialysis, phosphorus before dialysis, phosphorus after dialysis, potassium before dialysis, and potassium after dialysis) and dialysis parameter information data (including; dialysis date, dialysis duration, blood flow rate, dialysate flow rate, body weight after the last dialysis, body weight before dialysis, body weight after dialysis, and urea clearance rate of a dialyzer) through manual input or automatic acquisition and store the above data information into the storage.

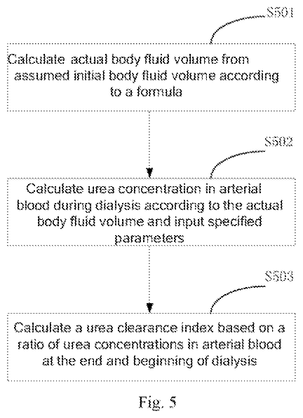

In a specific embodiment of the present application, the processor for calculating is specifically configured to calculate body fluid volume according to the most recent assay data; calculate urea concentration at the end of dialysis according to the calculated body fluid volume, dialysis time, removal volume of water, blood flow, and urea clearance rate of a dialyzer; and calculate the urea clearance index based on a ratio of urea concentration at the end and beginning of dialysis. In the processor for calculating, specific execution steps of a calculation process of the algorithm model shown in FIG. 5 are as follows;

S501; actual body fluid volume is calculated from assumed initial body fluid volume according to the formula, and various input parameters required for the calculation can be found in Table 1.

S502: urea concentration in arterial blood during dialysis is calculated according to the actual body fluid volume and input specified parameters; and various input parameters required for the calculation can be found in Table 2.

As actual body fluid volume is known, at the beginning of dialysis (t=0), water content distributed in each of visceral organs with high or low blood flow at t+1 (namely, 1 min after the dialysis starts) can be calculated. In combination with urea concentration in arterial blood and blood volume flowing out from the heart at the beginning of dialysis, area concentration in a visceral organ with, high or low blood flow at t+1 (namely, 1 min after the dialysis starts) can be calculated by a formula. Arterial urea concentration at t+1 (namely, 1 min after the dialysis starts) is calculated from the urea concentration at the beginning of dialysis, urea clearance rate of a dialyzer, and the blood volume flowing out from the heart.

That is, after this calculation is completed, urea concentration in visceral organs with high or low blood flow and arterial blood at t+1 (namely, 1 min after the dialysis starts) can be obtained, and the same method can be used to calculate urea concentration in each visceral organ at 2 min after the dialysis starts. Urea concentration in each visceral organ is calculated continuously and sequentially after every 1 min interval until urea concentration in visceral organs with high and low blood flow and arterial blood at the end of dialysis are obtained.

S503, the urea clearance Index is calculated based on a ratio of urea concentration in arterial blood at the end and beginning of dialysis.

Urea concentration at the beginning of dialysis is assumed to be 1 mg/mL, and urea concentration at the end of dialysis is calculated based on dialysis time, removal volume of water, blood flow, and urea clearance rate of a dialyzer; a ratio of urea concentration before and after dialysis is obtained; and finally, the ratio of urea concentration before and after dialysis is substituted into the Daugirdas formula to obtain a Kt/V value.

It should be noted that the algorithm module is configured to calculate adequacy result of the dialysis according to a regimen of each dialysis, so as to ensure the optimal comfort for a patient after each dialysis and finally realize ideal dialysis adequacy.

In summary, the present application discloses an adequacy assessment method and system. The method includes: acquiring biochemical test data, and extracting the most recent assay data recorded in periodic biochemical assays; calculating body fluid volume according to the most recent assay data of periodic assay records from the biochemical test data; calculating a urea clearance index according to the calculated body fluid volume and designated parameters; and outputting the urea clearance index for one adequacy assessment by display. One embodiment of the present application acquires biochemical test data of a patient before dialysis at one time to realize automatic assessment of each dialysis, which does not rely on multiple biochemical tests of blood sampling before and after HD and does not increase the blood loss burden of a patient due to repeated blood sampling. The dialysis adequacy can be monitored in real time and a treatment regimen can be adjusted during a dialysis process, without relying on a dialyzer and peripheral accessory devices. The embodiment is suitable for ail kinds of dialyzers to assess dialysis regimens and treatment effects on patients, A system with the same inventive concept as the above method includes: an input device, a processor for calculating, and an output device. The input device is configured to acquire biochemical test data and extract the most recent assay data of periodic assay records from the biochemical test data; the processor for calculating is configured to calculate body fluid volume according to the most recent assay data and thus calculate a urea clearance index according to the calculated body fluid volume and designated parameters; and the output device is configured to output the urea clearance index for one adequacy assessment by display. The input device is configured for medical staff to input or a computer to automatically acquire clinical data of patients; the processor for calculating is configured to process received clinical data of the patients and calculate through an assessment algorithm to obtain dialysis assessment result of the patients; and the output device is configured to output the adequacy assessment result. In the embodiment of the present application, the processor for calculating is configured to calculate adequacy result of the dialysis according to a regimen of each dialysis, so as to ensure the optimal comfort for a patient after each dialysis and finally realize ideal dialysis adequacy. Compared with the prior art, this application achieves real-time automatic assessment of dialysis adequacy through an algorithm software system, which does not rely on biochemical tests before and after dialysis and brand apparatus of dialysis device.

The processor for calculating is configured to: according to periodic assay data and dialysis data on the day of the assay, acquire blood urea concentration, dialysis time, removal volume of water, blood flow, and urea clearance rate at the beginning and end of dialysis; set an assumed value for initial body fluid volume, and after the dialysis starts, calculate urea concentration per unit time until the dialysis ends to obtain urea concentration at the body fluid volume; and continuously adjust the assumed value of body fluid volume, so that calculated urea concentration is consistent with urea concentration tested at the end of the dialysis to obtain actual body fluid volume.

The processor for calculating is configured to: according to known, actual body fluid volume at the beginning of dialysis, obtain water volume distributed in visceral organs with high and low blood flow; and according to removal volume of water per unit time, derive water volume in each visceral organ after each unit time; and obtain urea concentration in arterial blood at the beginning of dialysis through actual test, assuming that urea concentration in various visceral organs and arterial blood at the beginning of dialysis are consistent, and continuously and sequentially calculate urea concentration in various visceral organs and blood after each unit time according to the following formula until urea concentration in visceral organs with high, and low blood flow and arterial blood at the end of dialysis are obtained:

$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$

$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$

$${C}_{A} ( t + 1 ) = \frac{{Q}_{A} \times {C}_{A} ( t ) - K \times {C}_{A} ( t )}{{Q}_{A}}$$

wherein C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

Those skilled in the art can understand that a relevant hardware can be instructed through computer programs to implement all or part of processes in the method according to the above embodiments, and the programs can be stored in a computer-readable storage medium and the like. The computer-readable storage medium may be a magnetic disk, an optical disk, a read-only memory (ROM), a random access memory (RAM), or the like.

The above merely describes preferred specific implementations of the present application, but a protection scope of the present application is not limited thereto. Any person skilled in the art can easily conceive modifications or replacements within the technical scope of the present application, and these modifications or replacements shall fall within the protection scope of the present application.

The foregoing descriptions of specific exemplary embodiments of the present application have been presented for purposes of illustration and description. They are not intended to be exhaustive or to limit the application to the precise forms disclosed, and obviously many modifications and variations are possible in light of the above teachings. The exemplary embodiments were chosen and described in order to explain certain principles of the application and their practical application, to thereby enable otters skilled in the art to make and utilize various exemplary embodiments of the present application, as well as various alternatives and modifications thereof. It is intended that the scope of the application be defined by the Claims appended hereto and their equivalents.

1. An adequacy assessment method, comprising the following steps:
acquiring biochemical test data, and extracting the most recent assay data of periodic assay records from the biochemical test data;
calculating body fluid volume according to the most recent assay data;
calculating a urea clearance index according to the calculated body fluid volume and designated parameters; and
outputting the urea clearance index for one adequacy assessment by display.

2. The adequacy assessment method according to claim 1, wherein the method further comprises: storing adequacy assessment result for each dialysis and corresponding diagnosis and treatment data in a database.

3. The adequacy assessment method according to claim 1, wherein the acquiring biochemical test data comprises: inputting biochemical test data through an input interface or automatically acquiring biochemical test data from a medical system of biochemical test data.

4. The adequacy assessment method according to claim 1, wherein the calculating body fluid volume according to the most recent assay data comprises:
according to periodic assay data and dialysis data on the day of the assay, acquiring blood urea concentration, dialysis time, removal volume of water, blood flow, and urea clearance rate at the beginning and end of dialysis;
setting an assumed value for initial body fluid volume, and after the dialysis starts, calculating urea concentration per unit time until the dialysis ends to obtain urea concentration at the body fluid volume; and
continuously adjusting the assumed value of body fluid volume, so that calculated urea concentration is consistent with urea concentration tested at the end of the dialysis to obtain actual body fluid volume.

5. The adequacy assessment method according to claim 1 or wherein the calculating a urea clearance index according to the calculated body fluid volume and designated parameters comprises:
according to known actual body fluid volume at the beginning of dialysis, obtaining water volume distributed in visceral organs with high and low blood flow; and according to removal volume of water per unit time, deriving water volume in each visceral organ after each unit time; and
obtaining urea concentration in arterial blood at the beginning of dialysis through actual test, assuming that urea concentration in various visceral organs and arterial blood at the beginning of dialysis are consistent, and continuously and sequentially calculating urea concentration in various visceral organs and blood after each unit time according to the following formula until urea concentration in visceral organs with high and low blood flow and arterial blood at the end of dialysis are obtained:
$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$
$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$
$${C}_{A} ( t + 1 ) = \frac{{Q}_{A} \times {C}_{A} ( t ) - K \times {C}_{A} ( t )}{{Q}_{A}}$$
wherein C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

6. The adequacy assessment method according to claim 5, wherein the urea clearance index is calculated based on a ratio of urea concentration in arterial blood at the end and beginning of the dialysis.

7. An adequacy assessment system, wherein the system comprises: an input device, a processor for calculating, and an output device, wherein
the input device is configured to acquire biochemical test data and extract the most recent assay data of periodic assay records from the biochemical test data;
the processor for calculating is configured to calculate body fluid volume according to the most recent assay data and thus calculate a urea clearance index according to the calculated body fluid volume and designated parameters; and
the output device is configured to output the urea clearance index for one adequacy assessment by display.

8. The adequacy assessment system according to claim 7, wherein the system further comprises a storage configured to store adequacy assessment result for each dialysis and corresponding diagnosis and treatment data.

9. The adequacy assessment system according to claim 7, wherein the input device is configured to input biochemical test data through an input interface or automatically acquire biochemical test data from a medical system of biochemical test data.

10. The adequacy assessment system according to claim 7, wherein the processor for calculating is configured to calculate body fluid volume according to the most recent assay data; calculate urea concentration at the end of dialysis according to the calculated body fluid volume, dialysis time, removal volume of water, blood flow, and urea clearance rate of a dialyzer; and calculate the urea clearance index based on a ratio of urea concentration in arterial blood at the end and beginning of dialysis.

11. The adequacy assessment method according to claim 4, wherein the calculating a urea clearance index according to the calculated body fluid volume and designated parameters comprises:
according to known actual body fluid volume at the beginning of dialysis, obtaining water volume distributed in visceral organs with high and low blood flow; and according to removal volume of water per unit time, deriving water volume in each visceral organ after each unit time; and
obtaining urea concentration in arterial blood at the beginning of dialysis through actual test, assuming that urea concentration in various visceral organs and arterial blood at the beginning of dialysis are consistent, and continuously and sequentially calculating urea concentration in various visceral organs and blood after each unit time according to the following formula until urea concentration in visceral organs with high and low blood flow and arterial blood at the end of dialysis are obtained:
$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$
$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$
$${C}_{A} ( t + 1 ) = \frac{{Q}_{A} \times {C}_{A} ( t ) - K \times {C}_{A} ( t )}{{Q}_{A}}$$
wherein C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

12. The adequacy assessment method according to claim 11, wherein the urea clearance index is calculated based on a ratio of urea concentration in arterial blood at the end and beginning of the dialysis.

13. The adequacy assessment system according to claim 10, the processor for calculating is configured to:
according to periodic assay data and dialysis data on the day of the assay, acquire blood urea concentration, dialysis time, removal volume of water, blood flow, and urea clearance rate at the beginning and end of dialysis;
set an assumed value for initial body fluid volume, and after the dialysis starts, calculate urea concentration per unit time until the dialysis ends to obtain urea concentration at the body fluid volume; and
continuously adjust the assumed value of body fluid volume, so that calculated urea concentration is consistent with urea concentration tested at the end of the dialysis to obtain actual body fluid volume.

14. The adequacy assessment system according to claim 7, the processor for calculating is configured to:
according to known actual body fluid volume at the beginning of dialysis, obtain water volume distributed in visceral organs with high and low blood flow; and according to removal volume of water per unit time, derive water volume in each visceral organ after each unit time; and
obtain urea concentration in arterial blood at the beginning of dialysis through actual test, assuming that urea concentration in various visceral organs and arterial blood at the beginning of dialysis are consistent, and continuously and sequentially calculate urea concentration in various visceral organs and blood after each unit time according to the following formula until urea concentration in visceral organs with high and low blood flow and arterial blood at the end of dialysis are obtained:
$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$
$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$
$${C}_{A} ( t + 1 ) = \frac{{Q}_{A} \times {C}_{A} ( t ) - K \times {C}_{A} ( t )}{{Q}_{A}}$$
wherein C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.

15. The adequacy assessment system according to claim 13, the processor for calculating is configured to:
according to known actual body fluid volume at the beginning of dialysis, obtain water volume distributed in visceral organs with high and low blood flow; and according to removal volume of water per unit time, derive water volume in each visceral organ after each unit time; and
obtain urea concentration in arterial blood at the beginning of dialysis through actual test, assuming that urea concentration in various visceral organs and arterial blood at the beginning of dialysis are consistent, and continuously and sequentially calculate urea concentration in various visceral organs and blood after each unit time according to the following formula until urea concentration in visceral organs with high and low blood flow and arterial blood at the end of dialysis are obtained:
$${C}_{H} ( t + 1 ) = \frac{{C}_{A} ( t ) \times {Q}_{H} - {C}_{H} ( t ) \times {Q}_{H} + {C}_{H} ( t ) \times {V}_{H} ( t )}{{V}_{H} ( t + 1 )}$$
$${C}_{L} ( t + 1 ) = \frac{{C}_{A} ( t ) \times {Q}_{L} - {C}_{L} ( t ) \times {Q}_{L} + {C}_{L} ( t ) \times {V}_{L} ( t )}{{V}_{L} ( t + 1 )}$$
$${C}_{A} ( t + 1 ) = \frac{{Q}_{A} \times {C}_{A} ( t ) - K \times {C}_{A} ( t )}{{Q}_{A}}$$
wherein C_{H}(t) is urea concentration in visceral organs with high blood flow at time t, C_{L}(t) is urea concentration in visceral organs with low blood flow at time t, C_{A}(t) is urea concentration in arterial blood at time t, Q_{H} is blood flow in visceral organs with high blood flow, Q_{L} is blood flow in visceral organs with low blood flow, Q_{A} is blood volume flowing out from the heart, V_{H}(t) is water volume in visceral organs with high blood flow at time t, V_{L}(t) is water volume in visceral organs with low blood flow at time t, and K is urea clearance rate of a dialyzer.
